(19) United States
(12) Patent Application Publication
(10) Pub. No.: US 2015/0374325 A1
(43) Pub. Date: Dec. 31, 2015

(54) X-RAY DIAGNOSTIC APPARATUS

(71) Applicants: Kabushiki Kaisha Toshiba, Minato-ku (JP); Toshiba Medical Systems Corporation, Otawara-shi (JP)

(72) Inventors: Yoshinori SHIMIZU, Nasushiobara (JP); Masahiro OZAWA, Sakura (JP)

(73) Assignees: Kabushiki Kaisha Toshiba, Minato-ku (JP); Toshiba Medical Systems Corporation, Otawara-shi (JP)

(21) Appl. No.: 14/843,014

(22) Filed: Sep. 2, 2015

Related U.S. Application Data
(63) Continuation of application No. PCT/JP2014/055050, filed on Feb. 28, 2014.

(30) Foreign Application Priority Data
Mar. 4, 2013 (JP) 2013-041738

(51) Int. Cl. A61B 6/00

(52) U.S. Cl. CPC A61B 6/547; A61B 6/4441

(57) ABSTRACT

According to one embodiment, an X-ray diagnostic apparatus includes an X-ray tube which generates an X-ray, an X-ray detector which detects an X-ray generated by the X-ray tube, a support frame which supports the X-ray tube and the X-ray detector pivotally with respect to a plurality of movable axes, a storage which stores angular information represented by rotation angles about the movable axes, the angular information relates to at least one preset posture of the support frame, and a display which displays at least one first angle mark corresponding to the at least one posture on a clinical angle map represented by a coordinate system based on the movable axes defining a posture of the support frame.

### CROSS-REFERENCE TO RELATED APPLICATIONS

This application is a Continuation application of PCT Application No. PCT/JP2014/055050, filed Feb. 28, 2014 and based upon and claiming the benefit of priority from Japanese Patent Application No. 2013-041738, filed Mar. 4, 2013, the entire contents of all of which are incorporated herein by reference.

### FIELD

Embodiments described herein relate generally to an X-ray diagnostic apparatus.

### BACKGROUND

In some cases, an X-ray diagnostic apparatus needs an operation for moving a support, such as a C-arm, to change a direction of observation. The operation is performed by a manual input by a user utilizing an operation portion.

To facilitate an operation or work relating to a change of an observation position, a variety of techniques have been proposed. For example, Patent Literature 1 (Jpn. Pat. Appin. KOKAI Publication No. 2005-245502) discloses a technique for facilitating positioning of an X-ray tube and an X-ray detector constituting an imaging portion of an X-ray radiography diagnostic apparatus with respect to a subject under examination to reproduce an optimal observation direction (that is, an imaging direction). This technique is referred to as auto-positioning. The auto-positioning is an auxiliary function to place a support or table in a desired posture.

Patent Literature 2 (Jpn. Pat. Appin. KOKAI Publication No. 2006-262989) discloses a function for automatically reproducing switch of imaging programs or auto-positioning in sequence in an actual medical examination (such as routine examination), in which an observation direction is determined in advance, by registering sequences of the medical examination in advance. This function is referred to as a sequence auto-reproducing function.

Patent Literature 3 (Jpn. Pat. Appin. KOKAI Publication No. 8-289885) discloses a technique for displaying a list of imaging angles stored by the auto-positioning function on a display, when reproducing an imaging angle by auto-positioning.

The auto-positioning requires an operation of inputting a positioning number by a user and an operation of selecting from a number of candidate angles. A positioning number is a number tied to each of registered positions. A support or table is positioned at a desired position (that is, a desired position is reproduced) by inputting the positioning number by the user.

The user definitely recognizes a posture (angle) of the support or table to be reproduced, but does not necessarily remember the positioning number tied to the posture.

Therefore, each time the user selects a positioning, he or she must check a target posture displayed on the monitor. This check operation is troublesome for the user. Hence, there is a demand for a technique for easily positioning a support in a desired posture.

### BRIEF DESCRIPTION OF DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing a configuration example of an X-ray diagnostic apparatus according to a first embodiment.

FIG. 2 is a diagram showing an example of a coordinate system relating to drive of a support frame.

FIG. 3 is a flowchart of a clinical angle setting process by an X-ray diagnostic apparatus according to the first embodiment.

FIG. 4 is a diagram showing an example of a clinical angle map presented to a user in the clinical angle setting process.

FIG. 5 is a block diagram showing a cooperative relationship among a system controller, a display apparatus and interface circuitry, and a driving apparatus.

FIG. 6 is a diagram showing another example of a clinical angle map.

FIG. 7 is a diagram showing an example of a display of auxiliary information displayed along with a target posture mark.

FIG. 8 is a diagram showing an example of a display of auxiliary information displayed along with a target posture mark.

FIG. 9 is a diagram showing an example of a clinical angle map presented to a user in the clinical angle setting process.

FIG. 10 is a block diagram showing a cooperative relationship among a system controller, a display apparatus and an interface circuitry, and a driving apparatus.

FIG. 11 is a diagram showing one example of a display of auxiliary information displayed along with a target posture mark.

FIG. 12 is a diagram showing an example of a clinical angle map presented to a user in the clinical angle setting process.

FIG. 13 is a diagram showing an example of a clinical angle map presented to a user in the clinical angle setting process.

FIG. 14 is a diagram showing an example of a display of schematic images according to a fourth embodiment.

### DETAILED DESCRIPTION

In general, according to one embodiment, an X-ray diagnostic apparatus includes an X-ray tube, an X-ray detector, a support frame, a storage, and a display. The X-ray tube which generates an X-ray. The X-ray detector which detects an X-ray generated by the X-ray tube. The support frame which supports the X-ray tube and the X-ray detector pivotally with respect to a plurality of movable axes. The storage which stores angular information represented by rotation angles about the movable axes, the angular information relates to at least one preset posture of the support frame. The display which displays at least one first angle mark corresponding to the at least one preset posture on a clinical angle map represented by a coordinate system based on the movable axes defining a posture of the support frame.

An X-ray diagnostic apparatus according to the embodiments is described below.

### First Embodiment

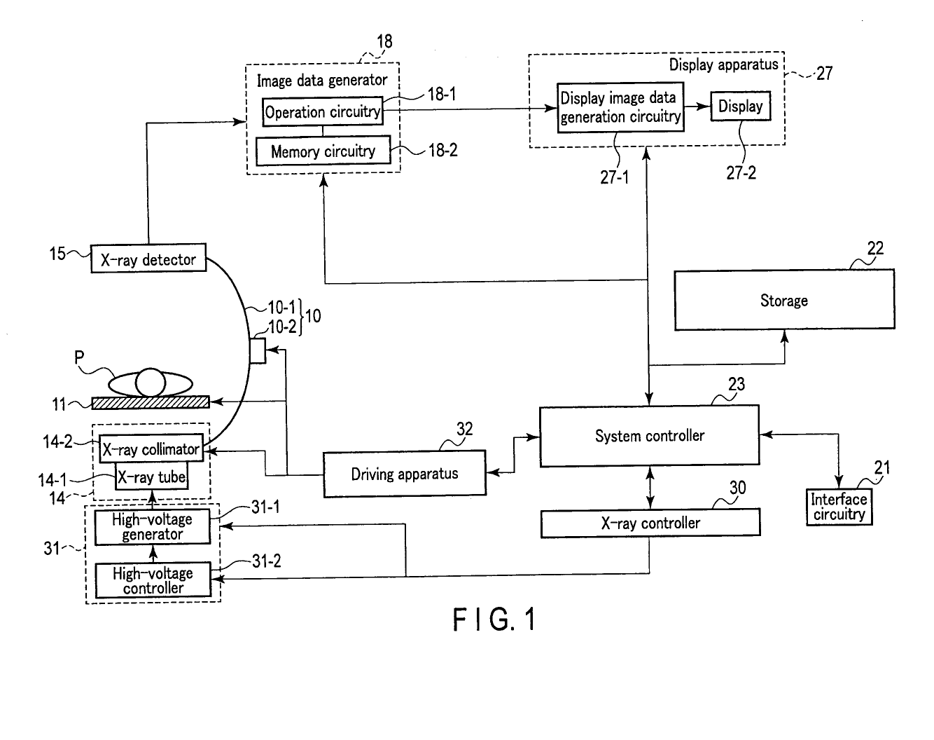

FIG. 1 is a block diagram showing a configuration example of an X-ray diagnostic apparatus according to a first embodiment. As show n in FIG. 1, an X-ray diagnostic apparatus 1 according to the first embodiment includes a support frame 10, a bed 11, an X-ray irradiator 14, an X-ray detector 15, an image data generator 18, an interface circuitry 21, an image storage 22, a system controller 23, a display apparatus 27, an X-ray controller 30, a high-voltage supply device 31, and a driving apparatus 32.

The support frame 10 is a mechanism which supports the X-ray irradiator 14 and the X-ray detector 15 pivotally with respect to a plurality of movable axes. Specifically, the support frame 10 includes a C-arm 10-1 and a base 10-2. The C-arm 10-1 supports the X-ray irradiator 14 and the X-ray detector 15 rotatably about an axis of rotation. The base 10-2 supports the C-arm 10-1 pivotally about an axis of pivot. An intersection of the axis of rotation and the axis of pivot is called an isocenter. Movable axes conceptually include axes of rotation and axes of pivot. The bed 11 is supported in a horizontal direction and a vertical direction. A subject P is laid on the bed 11.

The X-ray irradiator 14 includes an X-ray tube 14-1 and an X-ray collimator 14-2. The X-ray tube 14-1 is connected to a high-voltage generator 31-1. The X-ray tube 14-1 generates an X-ray upon receipt of supply of filament current and receipt of application of a high voltage from the high-voltage generator 31-1. The X-ray collimator 14-2 is attached to the X-ray tube 14-1. The X-ray collimator 14-2 restricts an irradiation field of an X-ray irradiated on the subject P.

The X-ray detector 15 detects an X-ray generated by the X-ray tube 14-1 and transmitted through the subject P.

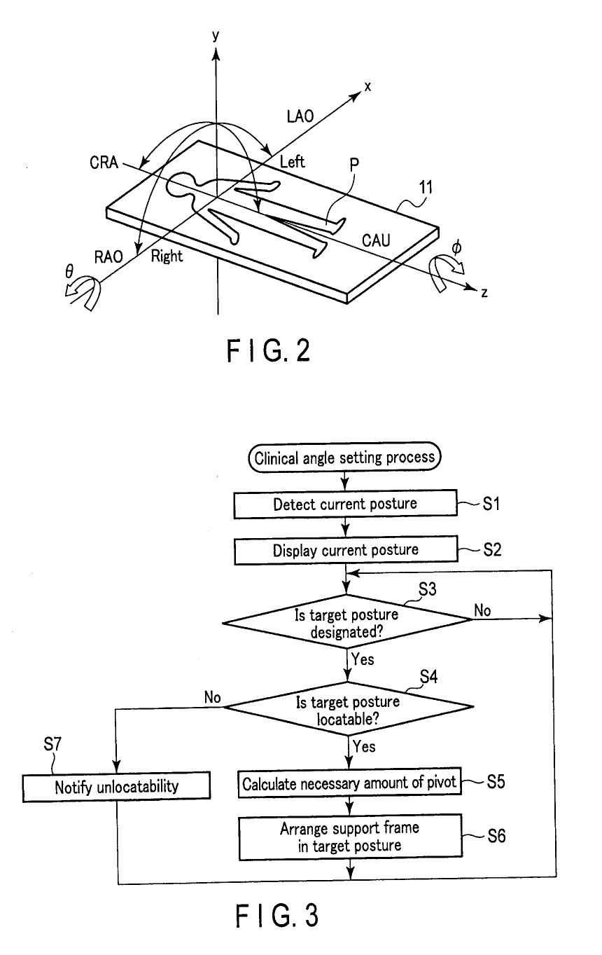

With reference to FIG. 2, a coordinate system (gantry coordinate system) relating to the support frame 10 will be explained. FIG. 2 is a diagram showing an example of the coordinate system relating to drive of the support frame 10. As shown in FIG. 2, an axis parallel to a long axis of the bed 11 is identified as a z-axis, an axis parallel to a short axis of the bed 11 is identified as an x-axis and an axis perpendicular to an upper surface of the bed 11 is identified as a y-axis. The x-axis, the y-axis and the z-axis constitute an orthogonal coordinate system.

An axis connecting a focal point of the X-ray tube 14-1 and a center of a detector plane of the X-ray detector 15 is called a central axis of imaging. An inclination of the central axis of imaging relative to the x-axis is defined as a rotation angle φ and an inclination thereof relative to the z-axis is defined as a rotation angle θ. If the inclination of the central axis of imaging is parallel to the y-axis, the rotation angles are defined as a reference angle; for example, the rotation angle φ and the rotation angle θ are defined as 0 degrees. With reference to the reference angle, a direction of inclination of the central axis of imaging in a +x-axis direction is defined as LAO (Left Anterior Oblique View: a second oblique position), and a direction of inclination in a −x-axis direction is defined as RAO (Right Anterior Oblique View: a first oblique position). Further, with reference to the reference angle, a direction of inclination of the central axis of imaging in a −z-axis direction is defined as CRA (Cranial View: a cranial direction), and a direction of inclination in a +z-axis direction its defined as CAU (Caudal View: a caudal direction).

The image data generator 18 includes operation circuitry 18-1 and memory circuitry 18-2. The operation circuitry 18-1 reads data in units of lines from the X-ray detector 15, and generates image data, such as perspective image data and photographic image data, based on the read data. The operation circuitry 18-1 associates the generated image data with imaging conditions supplied from the system controller 23. For example, a posture of the support frame 10 at a time of X-ray radiography may be an imaging condition. The posture of the support frame 10 specifically means an orientation of the C-arm 10-1 in the xyz coordinate system. In other words, the posture of the support frame 10 means a relative orientation of the C-arm 10-1 with respect to the bed 11. The memory circuitry 18-2 stores the image data generated by the operation circuitry 18-1.

The interface circuitry 21 is connected to the system controller 23 and receives inputs of various operations for the X-ray diagnostic apparatus 1. For example, the interface circuitry 21 may be a touch panel, a control panel, a foot switch, a joystick, or the like. The interface circuitry 21 of the first embodiment may be a touch panel. Thus, in the first embodiment, a touch panel is used as an input/output device.

The storage 22 is a memory device which stores X-ray image data etc. More specifically, the storage 22 stores X-ray image data in association with a posture of the support frame 10 taken when the X-ray image data is taken by X-ray radiography. Hereinafter, information on a posture is referred to as posture information. Specifically, posture information is defined as a rotation angle of the support frame 10.

The system controller 23 controls portions of the X-ray diagnostic apparatus 1 in an integrated manner.

The display apparatus 27 displays a variety of information. Specifically, the display apparatus 27 includes display image data generation circuitry 27-1 and a display 27-2. The display image data generation circuitry 27-1 converts the image data generated by the X-ray detector 15 to display image data to be displayed on the display 27-2. The display 27-2 displays the display image data generated by the display image data generation circuitry 27-1. The display apparatus 27 also displays a clinical angle map, which is described later.

The X-ray controller 30 controls the high-voltage supply apparatus 31 under control of the system controller 23.

The high-voltage supply apparatus 31 includes the high-voltage generator 31-1 and a high-voltage controller 31-2. The high-voltage generator 31-1 applies a high voltage and supplies a filament current to the X-ray tube 14-1 under control of the high-voltage controller 31-2.

The driving apparatus 32 individually rotates the support frame 10 and drives the bed 11 and the X-ray collimator 14-2 under control of the system controller 23.

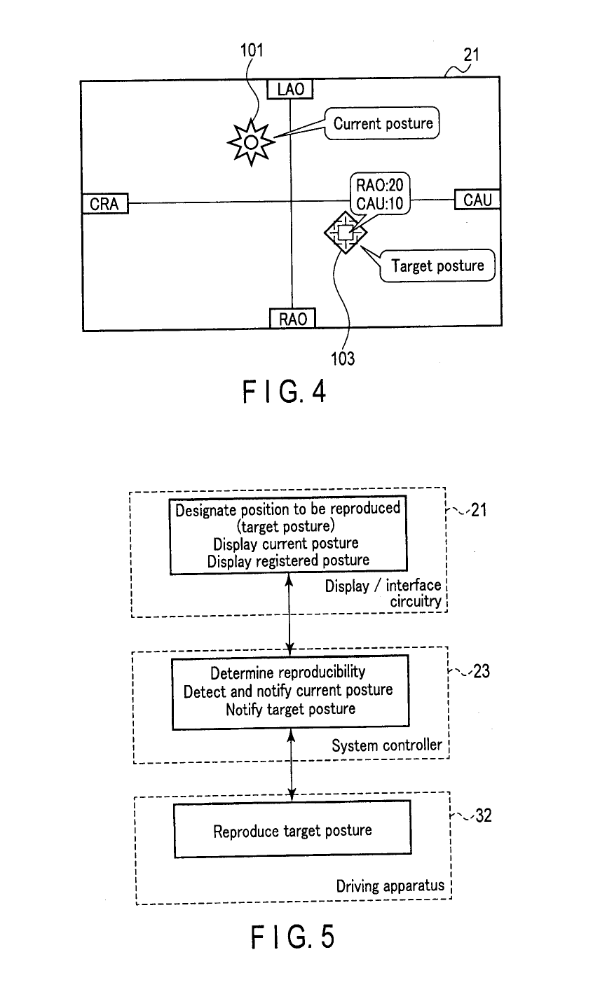

A clinical angle setting process by the system controller 23 of the X-ray diagnostic apparatus of the first embodiment will be described with reference to FIG. 3 to FIG. 5. FIG. 3 is a flowchart of a clinical angle setting process by the X-ray diagnostic apparatus according to the first embodiment. FIG. 4 is a diagram showing an example of a clinical angle setting screen presented to a user in the clinical angle setting process. FIG. 5 is a block diagram showing a correlation among the system controller 23, the display apparatus 27 and the Interface circuitry 21, and the driving apparatus 32.

First, the system controller 23 acquires posture information indicating a current posture of the support frame 10 from the driving apparatus 32 (Step S1). Hereinafter, posture information of the support frame 10 in the current state is referred to as current posture information. Specifically, the system controller 23 detects current posture information (rotation angle) of the support frame 10 based on an electric signal from a rotary encoder provided in the support frame 10.

Then, the system controller 23 causes the interface circuitry (touch panel) 21 to display the current posture information acquired in Step S1 (Step S2). For example, as shown in FIG. 4, the display apparatus 27 displays a mark corresponding to the current posture information (hereinafter referred to as a current posture mark) 101 on a clinical angle map. The clinical angle map is defined by the coordinate system based on the movable axes of the support frame 10, the movable axes defining the posture of the support frame 10. In FIG. 4, the clinical angle map is defined by an orthogonal coordinate system, in which the vertical axis is defined by the rotation angle φ and the horizontal axis is defined by the rotation angle θ. In other words, on the clinical angle map shown in FIG. 4, the vertical axis is defined by LAO and RAO and the horizontal axis is defined by CAU and CRA. The display apparatus 27 highlights coordinates of the current posture mark 101 on the clinical angle map based on the rotation angle corresponding to the current posture mark 101, and displays the current posture mark 101 at the specified coordinates. The user looks at the current posture mark 101 displayed on a plane of coordinates of the orthogonal coordinate system showing a clinical angle, and there by recognizes a clinical angle of the support frame 10 in the current state quantitatively and sensuously. Thus, the interface circuitry 21 functions as a display portion which causes posture information on the support frame 10 to be graphically displayed. More specifically, it functions as a display portion which causes current posture information to be displayed on the plane of coordinates of the orthogonal coordinate system, using a rotation angle of the support frame 10 about a movable axis as a parameter.

The system controller 23 determines whether target posture information indicating a target posture of the support frame 10 set in advance is designated or not (Step S3). In the first embodiment, the user operates the interface circuitry 21 to place a mark 103 corresponding to the target posture information on a desired position of the clinical angle map, thereby designating the target posture. Herein after, a mark corresponding to target posture information is referred to as a target posture mark. The target posture of the support frame 10 is designated by placing the target posture mark 103 in the desired position of the clinical angle map. As shown in FIG. 4, the display apparatus 27 preferably displays the current posture mark 101 and the target posture mark 103 in different forms to help the user to easily distinguish them. For example, the display apparatus 27 may display the current posture mark 101 and the target posture mark 103 as different marks. Alternatively, the display apparatus 27 may display the current posture mark 101 and the target posture mark 103 in different colors, shapes and patterns.

Further, the display apparatus 27 may display numerical values of rotation angles relating to the current posture mark 101 and the target posture mark 103 near the respective marks. For example, as shown in FIG. 4, the rotation angles (RAO: 20, CAU: 10) relating to the target posture mark 103 are displayed near the mark 103.

If Step S3 is branched to “NO”, the flow returns to Step S3. In other words, Step S3 is a step to wait until the target posture of the support frame 10 is designated.

In Step S3, if the system controller 23 determines that the target posture is designated (Step S3: YES), it determines whether the designated target posture is locatable or not (Step S4). More specifically, the system controller 23 determines whether the support frame 10 is locatable to the designated target posture based on a rotation angle in the current posture of the support frame 10 and a movable range of the support frame 10. If the rotation angle of the designated target posture is within the movable range of the support frame 10, the system controller 23 determines that the designated target posture is locatable. If the rotation angle of the designated target posture is not within the movable range of the support frame 10, the system controller 23 determines that the designated target posture is unlocatable. The movable range varies depending on the correlation between the current posture of the support frame 10 and the posture of the bed 11. Specifically, an angular range in which the support frame 10 mechanically interferes with the bed 11 is determined as unlocatable, and an angular range in which the support frame 10 does not mechanically interfere with the bed 11 is determined as locatable.

In Step S4, if the system controller 23 determines that the support frame 10 is unlocatable to the target posture (Step S4: NO), it notifies the user that the support frame 10 is “unlocatable to the input target posture” (Step S7). Thus, the system controller 23 functions as a notifying portion which notifies the user of unlocatability.

Specifically, the system controller 23 displays that, for example, “Support frame 10 is unlocatable to the target posture” on the display 27-2 in which the touch panel is provided. In addition to the notification by the display, the system controller 23 may notify that by sound or the like. After completing the process in Step S7, the system controller 23 proceeds to the process in Step S3.

If the system controller 23 determines that the support frame 10 is locatable to the target posture (Step S4: YES), it calculates an amount of pivot required to pivot the support frame 10 from the current posture to the target posture based on the current posture information and the target posture information (Step S5). The amount of pivot is defined by, for example, a difference between the rotation angle of the current posture and the rotation angle of the target posture.

The system controller 23 controls the driving apparatus 32 to locate the support frame 10 in the target posture (Step S6). After completing the process in Step S6, the system controller 23 proceeds to the process in Step S3.

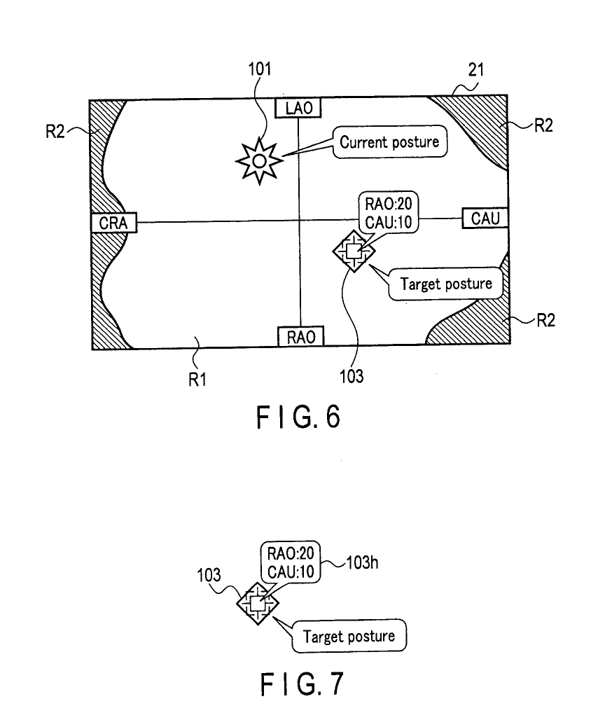

In the above description, the system controller 23 determines whether the support frame 10 is locatable to the target posture or not. However, the embodiment is not limited to the description. For Example, the display apparatus 27 may highlight, on the clinical angle map, an angular range in which the support frame 10 is movable or an angular range in which the support frame 10 is immovable. FIG. 6 is a diagram showing another display example of a clinical angle map. As shown in FIG. 6, the clinical angle map is divided into a movable range R1 and immovable range R2. The movable range R1 and the immovable range R2 are estimated by the system controller 23 as described above based on the rotation angle of the current posture and the movable range of the support frame 10. Specifically, an angular range in which the support frame 10 mechanically interferes with the bed 11 is presumed to be the immovable range R2, and an angular range in which the support frame 10 does not mechanically interfere with the bed 11 is presumed to be the movable range R1. Because the movable range R1 and the immovable range R2 are highlighted, the user can visually determine whether the support frame 10 is locatable in the target posture or not.

As described above, according to the first embodiment, it is possible to provide an X-ray diagnostic apparatus which allows the user to easily locate the support frame 10 and the bed 11 in a desired posture by an intuitive operation. In other words, the X-ray diagnostic apparatus according to this embodiment can reproduce a desired rotation angle, that is, a desired posture of the support frame 10.

Specifically, the X-ray diagnostic apparatus according to the first embodiment produces the following effects.

As described above with reference to FIG. 3 and FIG. 5, because the system controller 23 and the interface circuitry 21 cooperate, the user can intuitively perform a clinical angle setting process on the clinical angle setting screen shown in FIG. 4. Therefore, the user does not have to memorize or check the positioning number required to reproduce a desired posture (angle), and a troublesome operation can be omitted.

Accordingly, a manipulation operation time can be reduced and a manipulation operation can be performed smoothly, resulting in reduction of radiation exposure. Further, even if the user forgets the positioning number of a posture to be reproduced, the user can reproduce the posture to be reproduced by directly inputting the posture through the X-ray diagnostic apparatus of the first embodiment.

### Applied Example

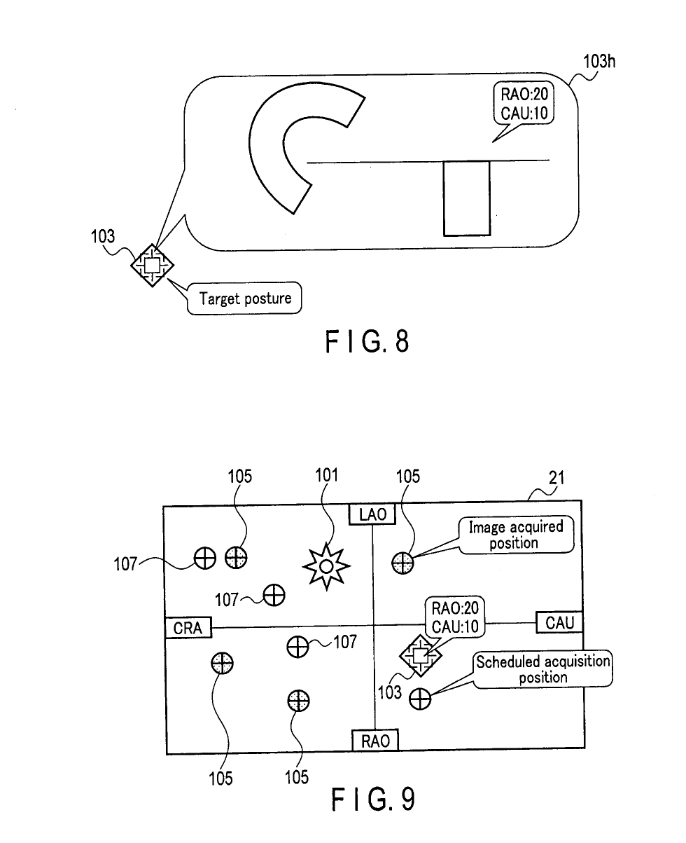

FIG. 7 and FIG. 8 are diagrams each showing an example of a display of auxiliary information displayed along with the target posture mark 103 by the display apparatus 27.

In the applied example, when the user clicks the target posture mark 103 indicated on the clinical angle map through the interface circuitry 21, the system controller 23 causes auxiliary information (rotation angle) 103h, which quantitatively indicates a clinical angle relating to the target posture mark 103, to be displayed as shown in FIG. 7. As shown in FIG. 8, the display apparatus 27 may display a schematic illustration schematically expressing the support frame 10 in a posture corresponding to the target posture mark 103 as auxiliary information 103h. The schematic illustration 103h may preferably express the support frame 10 viewed from the side of the interface circuitry 21, so that the user who operates the interface circuitry (touch panel) 21 can grasp a posture corresponding to the target posture mark 103. The schematic illustration 103h may include an illustration of the bed 11 as well as the support frame 10. In this case, the schematic illustration 103 schematically expresses a relative placement of the support frame 10 with respect to the table. The schematic illustration 103 and the target posture information relating to the target posture mark are stored by the storage 22 in association with each other. When the user designates the target posture mark 103, the display apparatus 27 reads a schematic illustration associated with target posture information relating to the designated mark 103 from the storage 22, and displays the read schematic illustration near the mark 103. Further, as shown in FIG. 8, the schematic illustration and the rotation angle may be displayed side by side.

The X-ray diagnostic apparatus of the applied example can present auxiliary information to the user when designating a target posture. Therefore, an operation for designating the target posture can be performed more easily and appropriately.

### Second Embodiment

An X-ray diagnostic apparatus according to a second embodiment is described below. To avoid duplication of explanations, differences from the first embodiment are explained below. Therefore, explanations for configurations, functions and effects common to those of the first embodiment are omitted accordingly.

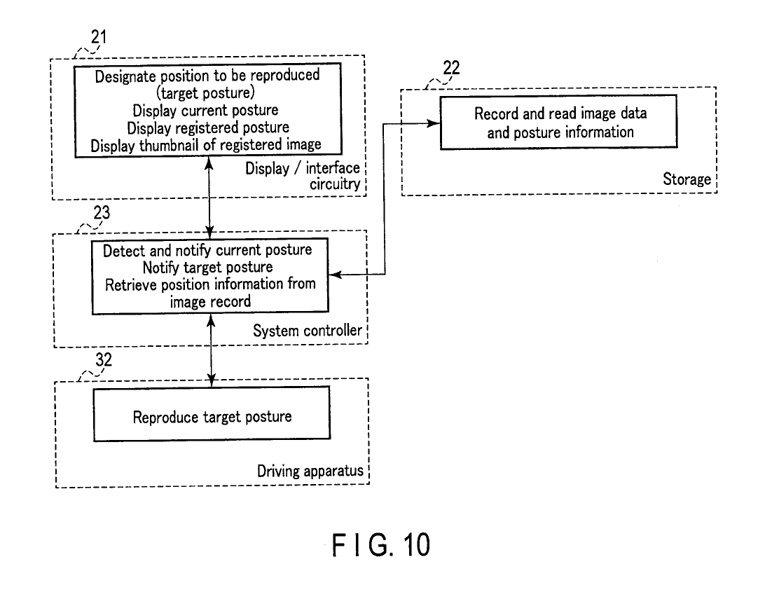

FIG. 9 is a diagram showing an example of a clinical angle setting screen presented to a user in the clinical angle setting process. FIG. 10 is a block diagram showing a cooperative relationship among the system controller 23, the display apparatus 27 and the interface circuitry 21, and the driving apparatus 32.

As described above, the storage 22 stores posture information of the support frame 10 (hereinafter referred to as imaging posture information), acquired when X-ray image data is actually taken by X-ray radiography, in association with the X-ray image data. The storage 22 stores a plurality of X-ray images respectively in association with imaging posture information of the support frame 10 acquired when the X-ray image is taken. As shown in FIG. 9, the display apparatus 27 of the second embodiment displays marks corresponding to the respective imaging posture information (hereinafter referred to as imaging posture marks) 105 on a clinical angle map.

Further, the storage 22 stores posture information on posture of the support frame 10 scheduled for X-ray radiography (hereinafter referred to as scheduled imaging posture information). The storage 22 stores scheduled imaging posture information corresponding to at least one scheduled imaging posture. The display apparatus 27 displays, on the clinical angle map, at least one mark (hereinafter referred to as a scheduled imaging posture mark) 107 corresponding to the at least one scheduled imaging posture. If the user designates a desired scheduled imaging posture mark 107 from the at least one scheduled imaging posture mark 107 through the interface circuitry 21, the system controller 23 controls the driving apparatus 32 to locate the support frame 10 in the scheduled imaging posture corresponding to the designated scheduled imaging posture mark 107.

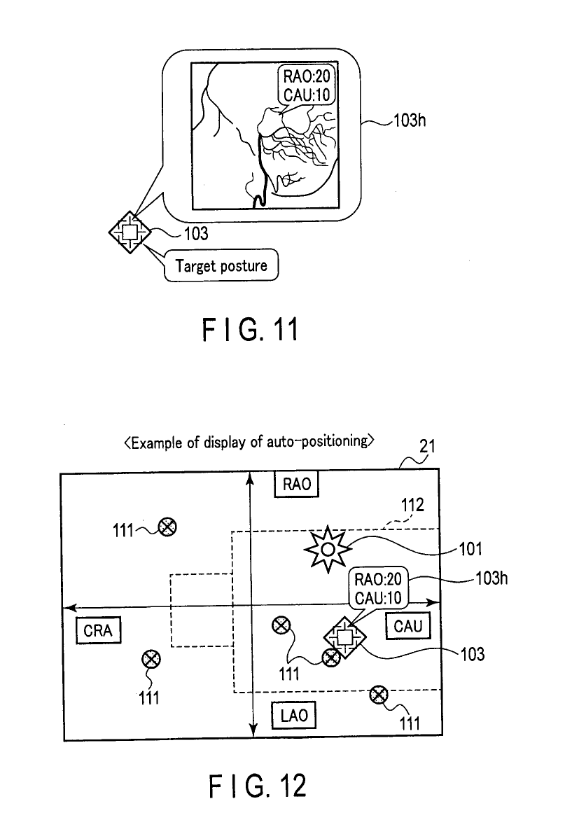

As in the applied example of the first embodiment, auxiliary in formation 103h may be displayed along with a target posture mark 103. FIG. 11 is a diagram showing an example of a display of auxiliary information displayed along with the target posture mark 103. If the user designates an imaging posture mark 105 through the interface circuitry 21, the system controller 23 reads X-ray image data associated with the designated imaging posture mark 105. The display apparatus 27 displays the X-ray image data as auxiliary information 103h along with angular information. The imaging posture mark 105 may be designated by directly designating the imaging posture mark 105 through the interface circuitry 21, or by placing the target posture mark 103 on the imaging posture mark 105. Thus, since auxiliary information for setting a target posture can be presented to the user, the target posture can be taken more easily and appropriately.

As described above, the X-ray diagnostic apparatus of the second embodiment produces the following effects in addition to the same effects as those of the X-ray diagnostic apparatus of the first embodiment.

Since the imaging posture mark 105 is displayed on the clinical angle map, the user can easily recognize a posture where image acquisition is actually performed. Therefore, it is ensured that X-ray radiography is properly performed in a scheduled posture. In particular, the embodiment produces great effect in a routine examination or the like.

In addition, the display apparatus 27 presents a number of X-ray images taken by X-ray radiography to the user in association with posture information on the support frame 10 at the time of X-ray radiography. Therefore, the X-ray diagnostic apparatus can easily reproduce the posture of the support frame 10 to take a desired X-ray image.

### Third Embodiment

An X-ray diagnostic apparatus according to a third embodiment is described below. To avoid duplication of explanations, differences from the first embodiment are explained below. Therefore, explanations for configurations, functions and effects common to those of the first embodiment are omitted accordingly.

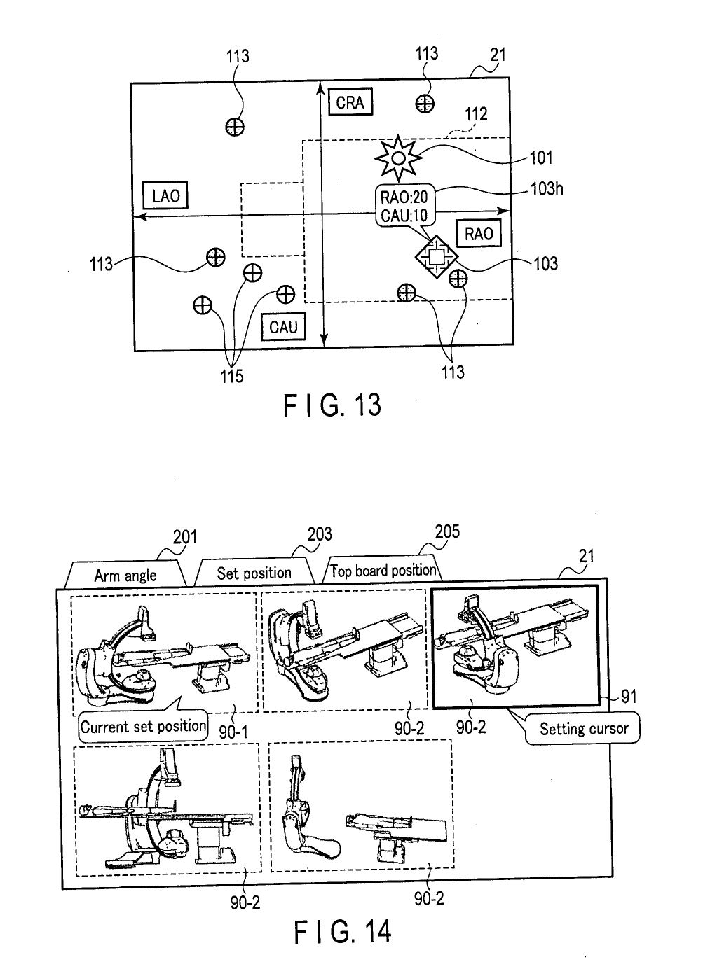

FIG. 12 and FIG. 13 are diagrams each showing an example of a clinical angle setting screen presented to a user in the clinical angle setting process.

A storage 22 of the third embodiment stores posture information on posture of a support frame 10 registered in advance (hereinafter referred to as registered posture information). The storage 22 stores registered posture information corresponding to at least one registered posture. A display apparatus 27 displays, on a clinical angle map, at least one mark (hereinafter referred to as a registered posture mark) 111 respectively corresponding to at least one registered posture. If the user designates a desired registered posture mark 111 from the at least one registered posture mark 111 through interface circuitry 21, a system controller 23 controls a driving apparatus 32 to locate the support frame 10 in the registered posture corresponding to the designated registered posture mark 111.

As shown in FIG. 12, registered posture marks 111 are displayed on a clinical angle map. Therefore, the user can easily locate the support frame 10 to a predetermined registered posture by moving a target posture mark 103 (dragging the mark on or near a registered posture mark 111) with reference to the registered posture marks. As shown in FIG. 12, the display apparatus 27 may pop up auxiliary information 103h along with the target posture mark 103.

The display apparatus 27 may display a mark (hereinafter referred to as the table mark) 112 representing the shape of a bed 11 (more specifically, a top board) on the clinical angle map, as shown in FIG. 12. Because the table mark 112 is displayed on the clinical angle map, the user can more clearly grasp a posture of the support frame 10 and a relative position with respect to the bed 11 represented by the respective marks.

As shown in FIG. 13, the display apparatus 27 may display a clinical angle map to assist an auto-angle function and a sequence auto-reproducing function. Specifically, the storage 22 stores registered posture information (hereinafter referred to as posture information for auto-angle) corresponding to a registered posture for auto-angle (hereinafter referred to as auto-angle posture). The display apparatus 27 displays a mark corresponding to the auto-angle posture (hereinafter referred to as a posture mark for auto-angle) 113 on the clinical angle map. The storage 22 stores registered posture information (hereinafter referred to as posture information for a sequence auto-reproducing function) corresponding to a registered posture for a sequence auto-reproducing function (hereinafter referred to as posture for a sequence auto-reproducing function). In the sequence auto-reproducing function, typically, a series of postures for a sequence auto-reproducing function are registered. The display apparatus 27 displays a mark 115 corresponding to the postures for a sequence auto-reproducing function (hereinafter referred to as a posture mark for a sequence auto-reproducing function) on the clinical angle map. At this time, the display apparatus 27 may preferably display the number of executed postures of all postures for the sequence auto-reproducing function on the clinical angular map. As a result, the user can clearly grasp a progress of examination sequence. Further, the display apparatus 27 may preferably display a standard order of locating the postures for the sequence auto-reproducing function to each of the posture marks for the sequence auto-reproducing function. Accordingly, the user can definitely grasp the order of locating the postures for the sequence auto-reproducing function.

According to the third embodiment, when the user designates a posture mark for the sequence auto-reproducing function through the interface circuitry 21, the system controller 23 switches between imaging programs based on the scheduled imaging information corresponding to the designated mark and sets target posture information relating to auto-positioning. Further, according to this embodiment, it is ensured that imaging in all scheduled postures in routine examination etc., is properly performed. As described above, according to the third embodiment, the auto-angle function and the sequence auto-reproducing function can be utilized more effectively.

Further, the display apparatus 27 may specifically display a posture which the support frame 10 cannot take on the clinical angle map.

As described above, the X-ray diagnostic apparatus of the third embodiment produces an effect of performing the clinical angle setting process more easily in addition to the same effects as those of the X-ray diagnostic apparatus of the first embodiment.

### Fourth Embodiment

An X-ray diagnostic apparatus according to a fourth embodiment is described below. To avoid duplication of explanations, differences from the first embodiment are explained below. Therefore, explanations for configurations, functions and effects common to those of the first embodiment are omitted accordingly.

In the above embodiments, the display apparatus 27 displays the clinical angle map to grasp a posture of the support frame 10 intuitively. However, the subject of display is not limited to the clinical angle map, as long as it allows a posture of the support frame 10 to be grasped intuitively. A display apparatus 27 of the fourth embodiment displays a schematic image which visually represents a posture of the support frame 10.

FIG. 14 is a diagram showing an example of a set position setting screen presented to a user in a set position setting process. As shown in FIG. 14, the display apparatus 27 of the fourth embodiment displays a plurality of schematic images 90. The schematic images 90 are classified into a schematic image 90-1 representing a posture of the support frame 10 in the current state and schematic images 90-2 representing predetermined postures set in advance. A storage 22 stores posture information corresponding to the schematic images 90-2, in association with the respective schematic images 90-2. The user designates a desired schematic image 90-2 from the schematic images 90-2 through the interface circuitry 21. For example, the user designates a desired schematic image 90-2 by placing a setting cursor 91 on the desired schematic image 90-2 through the interface circuitry 21. The system controller 23 controls a driving apparatus 32 to locate the support frame 10 in the posture corresponding to the designated schematic image 90-2.

In FIG. 14, a tab 201 is a tab to switch to an operation screen for setting a posture of a C-arm, a tab 203 is a tab to switch to an operation screen for setting “a set position”, and a tab 205 is a tab to switch to an operation screen for setting a position of a top board of a bed 11. A schematic image indicating a posture of the support frame 10 corresponding to each of the tabs is displayed in a window. The display apparatus 27 does not necessarily display a tab. In other words, all kinds of schematic images may be displayed in a single window.

As described above, the X-ray diagnostic apparatus of this embodiment includes an X-ray tube 14-1, an X-ray detector 15, the support frame 10, the storage 22 and the display apparatus 27. The X-ray to be 14-1 generates X-rays. The X-ray detector 15 detects an X-ray generated by the X-ray tube 14-1. The support frame 10 supports the X-ray tube 14-1 and the X-ray detector 15 pivotally with respect to a plurality of movable axes. The storage 22 stores angular information posture information) represented by rotation angles about a plurality of movable axes relating to at least one posture set in advance for the support frame 10. The display apparatus 27 displays at least on e first angle mark corresponding to the at least one posture on a clinical angle map represented by a coordinate system based on the movable axes which define a posture of the support frame 10.

Because of the configuration described above, the user can intuitively understand a posture of the support frame 10 to locate, and provide instructions for positioning the support frame 10 by an intuitive operation.

While certain embodiments have been described, these embodiments have been presented by way of example only, and are not intended to limit the scope of the inventions. Indeed, the novel embodiments described herein may be embodied in a variety of other forms; further more, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An X-ray diagnostic apparatus comprising:
an X-ray tube which generates an X-ray;
an X-ray detector which detects an X-ray generated by the X-ray tube;
a support frame which supports the X-ray tube and the X-ray detector pivotally with respect to a plurality of movable axes;
a storage which stores angular information represented by rotation angles about the movable axes, the angular information relates to at least one preset posture of the support frame; and
a display which displays at least one first angle mark corresponding to the at least one preset posture on a clinical angle map represented by a coordinate system based on the movable axes defining a posture of the support frame.

2. The X-ray diagnostic apparatus according to claim 1, further comprising:
a driving apparatus which drives the support frame; and
a controller which controls the driving apparatus to arrange the support frame in a posture corresponding to an angle mark of the at least one first angle mark designated by a user.

3. The X-ray diagnostic apparatus according to claim 2, wherein the controller determines whether the support frame is locatable in a posture corresponding to the designated angle mark based on a current posture and a movable range of the support, if determines that the support frame is not locatable in the posture corresponding to the designated angle mark, notifies that the support is not locatable in the posture, and if determines that the support is locatable in the posture corresponding to the designated angle mark, controls the driving apparatus to arrange the support frame in the posture corresponding to the designated angle mark.

4. The X-ray diagnostic apparatus according to claim 1, further comprising:
a detector which detects second angular information relating to a current posture of the support frame, wherein
the display displays a second angle mark corresponding to the second angular information on the clinical angle map along with the at least one first angle mark.

5. The X-ray diagnostic apparatus according to claim 4, wherein the display displays the first angle mark and the second angle mark in different forms.

6. The X-ray diagnostic apparatus according to claim 1, wherein the clinical angle map is an orthogonal two-dimensional coordinate having a horizontal axis defined by CRA/CAU and a vertical axis defined by LAO/RAO.

7. The X-ray diagnostic apparatus according to claim 1, wherein the display displays the at least one first angle mark and an X-ray image taken previously in a posture corresponding to the at least one first angle mark.

8. The X-ray diagnostic apparatus according to claim 7, wherein if a user designates an arbitrary angle mark from the at least one first angle mark, the display displays the designated arbitrary angle mark and an X-ray image taken previously in a posture corresponding to the designated arbitrary angle mark.

9. The X-ray diagnostic apparatus according to claim 1, wherein the display displays the at least one first angle mark and a schematic illustration representing the support in the posture corresponding to the at least one first angle mark.

10. The X-ray diagnostic apparatus according to claim 9, wherein if a user designates an arbitrary angle mark from the at least one first angle mark, the display displays the designated arbitrary angle mark and a schematic illustration representing the support in the posture corresponding to the designated arbitrary angle mark.

11. The X-ray diagnostic apparatus according to claim 1, wherein the display highlights an angular range in which the support is movable or immovable in the clinical angle map.

12. An X-ray diagnostic apparatus comprising:
an X-ray tube which generates an X-ray;
an X-ray detector which detects the X-ray generated by the X-ray tube;
a support frame which supports the X-ray tube and the X-ray detector pivotally with respect to a plurality of movable axes;
a driving apparatus which drives the support frame;
a storage which stores at least one schematic image, the schematic image visually represents at least one preset posture of the support frame;
a display which displays at least one schematic image; and
a controller which controls the driving apparatus to arrange the support frame in a posture corresponding to the schematic image of the at least one schematic mark designated by a user.
